SEQUENCE LISTING (3 sequences)

SEQ ID NO: 1 - protein, 546 aa, synthetic construct
MEFGLSWVFLVALFRGVQCQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTIKVLDLKNYIDKQLLPIINKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGHHHHHH

SEQ ID NO: 2 - DNA, 1641 bp, synthetic construct
  source 1..1641: mol_type = other DNA
atggagtttgggctgagctgggttttcctcgttgctctttttagaggtgtccagtgtcagaatatcacagaggagttctaccagtctacctgtagcgccgtgtccaagggatacctgagcgccctgaggaccggatggtatacatctgtgatcaccatcgagctgagcaacatcaaggagaacaagtgcaatggcacagacgccaaggtgaagctgatcaagcaggagctggataagtacaagaatgccgtgaccgagctgcagctgctgatgcagtccacccctgccacaaacaatcgggcccggagagagctgccaagattcatgaactataccctgaacaatgccaagaagaccaatgtgacactgagcaagaagcgcaagaggcgcttcctgggctttctgctgggagtgggctctgccatcgcaagcggagtggccgtgtgcaaggtgctgcacctggagggcgaggtgaacaagatcaagtccgccctgctgtctacaaacaaggccgtggtgagcctgtccaatggcgtgtccgtgctgaccatcaaggtgctggacctgaagaattacatcgataagcagctgctgcctatcatcaacaagcagtcttgtagcatctccaatatcgagacagtgatcgagttccagcagaagaacaataggctgctggagatcacccgcgagtttagcgtgaacgccggcgtgaccacaccagtgtccacatacatgctgaccaatagcgagctgctgtccctgatcaacgacatgcccatcaccaatgatcagaagaagctgatgtccaacaatgtgcagatcgtgcggcagcagtcttatagcatcatgtgcatcatcaaggaggaggtgctggcctacgtggtgcagctgcctctgtatggcgtgatcgacacaccatgctggaagctgcacacctctcccctgtgcaccacaaacacaaaggagggcagcaatatctgcctgacccggacagacagaggctggtactgtgataacgccggctccgtgtctttctttcctcaggccgagacctgcaaggtgcagagcaaccgggtgttctgtgacacaatgaacagcctgaccctgcccagcgaggtgaacctgtgcaatgtggacatctttaaccctaagtatgattgtaagatcatgacctccaagacagatgtgagctcctctgtgatcacaagcctgggcgccatcgtgtcctgctacggcaagaccaagtgtacagcctctaacaagaatagaggcatcatcaagaccttctccaacggctgtgactacgtgagcaataagggcgtggatacagtgtccgtgggcaacaccctgtactatgtgaataagcaggagggcaagtctctgtacgtgaagggcgagccaatcatcaacttctatgacccactggtgttccccagcgacgagtttgatgccagcatctcccaggtgaacgagaagatcaatcagtccctggcctttatcaggaagtccgatgagctgctgtctgccatcggaggatacatcccagaggcacctagggacggacaggcatatgtgcgcaaggatggcgagtgggtgctgctgagcacctttctgggcggccaccaccaccaccaccactga

SEQ ID NO: 3 - protein, 522 aa, synthetic construct
MQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGG

(19) United States
(12) Patent Application Publication
(10) Pub. No.: US 2024/0148856 A1
(43) Pub. Date: May 9, 2024

(54) RESPIRATORY SYNCYTIAL VIRUS VACCINE

(71) Applicant: EUBIOLOGICS CO., LTD., Seoul (KR)

(72) Inventors: Chan Kyu LEE, Chuncheon-si (KR); Jonathan F. LOVELL, Niagara Falls (CA); Yoon Hee WHANG, Chuncheon-si (KR); Woo Yeon HWANG, Chuncheon-si (KR); Hye Ji KIM, Chuncheon-si (KR); Min Chul PARK, Chuncheon-si (KR); Seok Kyu KIM, Seoul (KR); Wei-Chiao HUANG, Buffalo, NY (US); Da Hui HA, Chuncheon-si (KR)

(73) Assignee: EUBIOLOGICS CO., LTD., Seoul (KR)

(21) Appl. No.: 18/495,475

(22) Filed: Oct. 26, 2023

Related U.S. Application Data
(60) Provisional application No. 63/422,093, filed on Nov. 3, 2022.

(30) Foreign Application Priority Data
Nov. 23, 2022 (KR) 10-2022-0158440

(51) Int. Cl. A61K 39/155; A61P 31/14; C07K 1/18; C07K 1/22; C07K 7/00; C07K 14/005; C12N 5/071

(52) U.S. Cl. CPC A61K 39/155; A61P 31/14; C07K 1/18; C07K 1/22; C07K 7/00; C07K 14/005; C12N 5/0682; A61K 2039/55555

(57) ABSTRACT

Provided is a vaccine composition for preventing respiratory syncytial virus (RSV) infection, which is in the form of a liposome formulation including a RSV antigen, monophosphoryl lipid A (MLA), and/or a cobalt-porphyrin-phospholipid (CoPoP) conjugate. The vaccine composition exhibits excellent vaccine efficacy from a RSV antigen with enhanced immunogenicity and a combination of immune adjuvants for enhancing immune activity and antigen presentation.

### CROSS-REFERENCE TO RELATED APPLICATION

This application is based on and claims priority under 35 U.S.C. § 119 to U.S. Provisional Application No. 63/422,093 filed on Nov. 3, 2022 and Korean Patent Application No. 10-2022-0158440, filed on Nov. 23, 2022, in the Korean Intellectual Property Office, the disclosure of each of which is incorporated by reference herein in its entirety.

### REFERENCE TO AN ELECTRONIC SEQUENCE LISTING

The contents of the electronic sequence listing (Sequence_Listings_1183-0156PUS2.xml; Size: 5,740 bytes; and Date of Creation: Oct. 18, 2023) is herein incorporated by reference in its entirety.

### BACKGROUND

### 1. Field

The disclosure relates to a respiratory syncytial virus (RSV) vaccine composition including a RSV antigen such as RSVF-E2 (RSV fusion glycoprotein E2), monophosphoryl lipid A (MLA), and a cobalt-porphyrin-phospholipid (CoPoP) conjugate, and a preparation method and use thereof.

### 2. Description of the Related Art

With the increasing demand for vaccine safety, the development of subunit antigens have increased because structures and components thereof are defined and safe compared to those of live-attenuated or inactivated vaccines. However, subunit antigens exhibit higher stability but lower vaccine efficacy than live-attenuated or inactivated vaccines, and thus use of immune adjuvants in the development of vaccines has increased to enhance vaccine efficacy of subunit antigens. Immune adjuvants for medical purposes have been mainly used in the development of human vaccines to enhance immunogenicity of vaccines; however, applications of immune adjuvants have expanded to areas such as anticancer therapy and treatment of autoimmune disorders, since specific mechanisms of action of immune adjuvants have been determined and immunotherapy to treat diseases by controlling the immune system of the body has drawn attention.

Respiratory syncytial virus (RSV) is a common respiratory virus that spreads from early fall to early spring every year and causes upper and lower respiratory diseases in infants, the elderly, and immunocompromised patients. Infectivity of RSV is so high as to infect almost all infants by age two, and in particular, RSV infection in high-risk groups such as premature babies and those with chronic lung diseases or congenital heart diseases may lead to serious complications, resulting in high mortality rate. While RSV infection in adults produces only mild cold-like symptoms, severe infection may be developed in infants, people with weakened immune system, or the elderly. Despite these seriousness, no vaccine against RSV has been approved, unlike influenza, a respiratory virus against which vaccines have been approved.

Palivizumab (Synagis), a monoclonal antibody specific for fusion (F) protein of RSV is currently used to prevent lower respiratory tract infection by RSV. Palivizumab is mainly recommended for children less than two years of age who are at high risks for RSV infection, with 5 doses, which requires a high cost. In addition, vaccination of Palivizumab may not always lead to protective effect. Thus, there is an urgent need to develop an economical and effective RSV vaccine.

Conventional immune adjuvants such as aluminum and emulsions are non-immunostimulatory ingredients and thus do not directly stimulate immune cells but act to deliver antigens to the immune system effectively by binding the antigens. Various other immune adjuvants enhance immune activity by directly activating innate immune receptors such as toll like receptors (TLR). Effects of vaccine on immune enhancement may be strengthened by combination of immune adjuvants for immune activity increase and a formulation for improvement of antigen delivery efficiency.

Therefore, we have conducted studies on a RSV vaccine to effectively suppress or prevent RSV infection and developed a RSV vaccine having excellent vaccine efficacy based on a RSV antigen with increased immunogenicity, immune adjuvants enhancing immune responses, and a formulation providing improved antigen presentation.

### SUMMARY

Provided is a vaccine composition for treating or preventing respiratory syncytial virus (RSV) infection.

Provided is a method of preparing a vaccine composition for treating or preventing RSV infection.

Provided is a method of treating or preventing RSV infection.

Additional aspects will be set forth in part in the description which follows and, in part, will be apparent from the description, or may be learned by practice of the presented embodiments of the disclosure.

An aspect of the disclosure provides a vaccine composition for preventing respiratory syncytial virus (RSV) infection, which is in the form of a liposome formulation comprising a RSV antigen, monophosphoryl lipid A (MLA), and/or a cobalt-porphyrin-phospholipid (CoPoP) conjugate.

The vaccine composition according to an aspect of the present disclosure may include a RSV antigen and either MLA or CoPoP or both MLA and CoPoP.

As used herein, the term “vaccine composition” may be used interchangeably with “immunogenic composition”. The vaccine composition refers to a composition of substances suitable for administration to a human or animal subject to induce an immune response specific to a pathogen (for example, RSV). The vaccine composition includes one or more antigens such as whole purified viruses or antigenic subunits, for example, polypeptides thereof, or antigenic epitopes.

Respiratory syncytial virus (RSV), belonging to the Paramyxoviridae family, the genus Pneumovirus, is a lipoprotein-enveloped virus. The lipoproteins of the RSV envelope include F protein (fusion glycoprotein), G protein (attachment glycoprotein), and SH (small hydrophobic) protein. The F and G proteins are responsible for binding to a cell membrane, and the F protein plays an essential role in the entry of the RSV.

The vaccine composition according to an aspect of the present disclosure is a vaccine composition in the form of a liposome formulation comprising monophosphoryl lipid A (MLA), as an immune adjuvant, in a bilayer membrane of a liposome formed of a hydrophilic core and the bilayer membrane surrounding the core so that the antigen is delivered via the liposome and the immuno adjuvant therein enhances immune responses, thereby increasing the efficacy as the RSV vaccine.

As used herein, the term “lipid A” refers to a lipid consisting of two glucosamines such as carbohydrates or sugars and acyl chains bound thereto. In general, lipid A includes one phosphate group attached to each glucosamine. Lipid A may be tri-, tetra-, penta-, hexa-, or hepta-acylated, depending on the number of acyl chains. Four acyl chains directly attached to glucosamine may be beta hydroxyacyl chains, each formed of 10 to 16 carbons in length, and two additional acyl chains may be attached to beta hydroxy groups.

As used herein, the term “monophosphoryl lipid A (MLA)” may be used interchangeably with MPL, MPLA, MPL-A, or Escherichia coli MLA (EcML), and may refer to lipid A in which only one of the two glucosamines has a phosphate group attached thereto. The MLA may be tri-, tetra-, penta-, hexa-, or hepta-acylated depending on the number of acyl chains, and may be, for example, 1-dephosphorylated lipid A, 1-dephosphorylated-pentaacyl lipid A, 1-dephosphorylated-tetraacyl lipid A, or a combination thereof. The term “1-dephosphorylated-lipid A” refers to a MLA in which a phosphate group at position 1 in the structure of lipid A is replaced by a hydroxy group, which may be hexa-acylated. The term may be used interchangeably with “1-dephosphorylated-hexaacyl lipid A” or “hexaacylated 1-dephosphorylated lipid A”. MLA, as a Toll like receptor-4 (TLR-4) agonist, is a part of lipopolysaccharide (LPS), an endotoxin present in outermembrane of Gram-negative bacteria. LPS is released from a bacterial membrane by a LPS binding protein (LBP), delivered to CD14, and then delivered to a TLR4-MD-2 complex on a cell surface to induce dimerization of the complex. The LPS-induced complex dimer leads to activation of signaling via two different pathways. The LPS-induced complex dimer delivers an immune response into a cell to activate NFκB, a transcription factor, via adaptor proteins, TIRAP and MyD88. In addition, the TLR4-MD-2 complex is absorbed into the cell to promote activation of late NFκB and IRF3 via adaptor proteins TRIF and TRAM, leading to secretion of Type I interleukin. Overactivity of NFκB causes shocks such as septicaemia and allergies. Although LPS is an excellent TLR4 agonist as described above, LPS itself is too toxic to be used as an immune adjuvant. Thus, only lipid A portion isolated from LPS is used as an immune adjuvant. Lipid A produced by E. coli has phosphate groups bound to two glucosamine backbones at positions 1 and 4, and four acyl chains directly bound to the glucosamine head group at positions 2, 3, 2′, and 3′. The other two acyl chains are bound to hydroxyl groups of acyl chains attached to the glucosamine head group at positions 2′ and 3′. In the mechanism of LPS acting as an endotoxin, a phosphate group bound to the glucosamine backbone at position 1 plays an important role. Unlike lipid A, MLA in which the above-described phosphate group is replaced by a hydroxyl group exhibits a strong activity as an immune adjuvant without any side effects.

GlaxoSmithKline (GSK) developed as an immune adjuvant MPL (extracted from Salmonella minnesota and chemically modified), a type of MLA. We developed E. coli Monophosphoryl Lipid A (EcML) having similar structure and functions to those of MLA (see Korean Patent No. 2019331). EcML is produced by E. coli genetically engineered to directly accumulate MLA on the cell membrane thereof, enabling high-quality MLA to be efficiently produced with a low cost.

The MLA may not include a sugar moiety. The sugar moiety may be 2-keto-3-deoxy-D-manno-octulosonate (Kdo). Kdo is a component of lipopolysaccharide (LPS) and is a conserved residue found in almost all LPSs.

The MLA may be present in the membranes of living bacteria, for example, outer membranes.

The MLA may be chemically synthesized or isolated from a MLA producing strain. The MLA producing strain may be, for example, wild-type or genetically engineered Escherichia coli (E. coli), and may be an E. coli KHSC0055 strain disclosed in Korean Patent No. 10-2019331.

In an embodiment of the present disclosure, the MLA may be included in an amount of about 0.0001 wt % to about 10 wt %, about 0.0001 wt % to about 5 wt %, about 0.0001 wt % to about 1 wt %, about 0.0001 wt % to about 0.5 wt %, about 0.0001 wt % to about 0.1 wt %, about 0.0001 wt % to about 0.05 wt %, about 0.001 wt % to about 10 wt %, about 0.001 wt % to about 5 wt %, about 0.001 wt % to about 1 wt %, about 0.001 wt % to about 0.5 wt %, about 0.001 wt % to about 0.1 wt %, or about 0.001 wt % to about 0.05 wt % based on a total weight of the vaccine composition, but is not limited thereto.

The vaccine composition according to an aspect of the present disclosure may include a cobalt-porphyrin-phospholipid conjugate to display the RSV antigen on the surface of the liposome more effectively, thereby providing improved antigen delivery and vaccine efficacy.

The use of CoPoP, which may also be referred to as CoPoP technology, takes advantage of the strong binding between a cobalt ion and a histidine-tag (His-tag) to display an antigen on the surface of a liposome. This approach may be implemented by CoPoP prepared by conjugating cobalt ion-containing porphyrins to phospholipid in a liposome, and a CoPoP liposome may be prepared by incorporating CoPoP into a lipid bilayer structure of a liposome by adding CoPoP to a liposome forming composition (See U.S. Pat. No. 11,207,421). If a histidine-tag is expressed as a part of a recombinant antigen, the antigen binds to a cobalt of the CoPoP containing liposome, thereby being displayed on the surface of the liposome. Such display of the antigen increases the probability of antigen absorption into an antigen-presenting cell (APC) and thus leads to high immunogenicity compared to the antigen display from simple mixing of the liposome and the antigen.

The porphyrin portion of the CoPoP conjugate may be porphyrins, porphyrin derivatives, porphyrin analogs, or combination thereof. Exemplary porphyrins may include hematoporphyrin, protoporphyrin, and tetraphenylporphyrin. Exemplary porphyrin derivatives may include pyropheophorbides, bacteriochlorophylls, chlorophyll A, benzoporphyrin derivatives, tetrahydroxyphenylchlorines, purpurins, benzochlorines, naphthochlorins, verdins, rhodins, ketochlorins, azachlorins, bacteriochlorins, triporphyrins, and benzobacteriochlorins. Exemplary porphyrin analogs may include expanded porphyrin family members (for example, texaphyrins, saphirins, and hexaphyrins) and porphyrin isomers (for example, porphycenes, inverted porphyrins, phthalocyanines, and naphthalocyanines). The cobalt-porphyrin may be, for example, vitamin B12 (cobalamin) or a derivative thereof.

The phospholipids in the CoPoP conjugate may refer to a lipid having a hydrophilic head with a phosphate group and hydrophobic tails derived from fatty acids, joined by a glycerol backbone. The phospholipid may include an acyl side chain having 6 to 22 carbon atoms. The phospholipid may include, for example, 1-palmitoyl-2-hydroxy-sn-glycero-3-phosphocholine, phosphatidylcholine, phosphatidylethanolamine, phosphatidylserine, phosphatidylinositol, or a combination thereof, but is not limited thereto.

In the CoPoP conjugate, the porphyrin may be bound to the glycerol group of the phospholipid by a carbon chain linker having 1 to 20 carbon atoms.

In addition, the vaccine composition according to an aspect of the present disclosure includes MLA and CoPoP, thereby providing enhanced immune response, effective display of the RSV antigen on the surface of the liposome, which leads to improved antigen delivery and vaccine efficacy.

In an embodiment of the present disclosure, the vaccine composition may further include saponin-based QS-21.

In an embodiment of the present disclosure, the vaccine composition may be a vaccine composition in the form of a liposome formulation including a RSV antigen, MLA, and QS-21.

In an embodiment of the present disclosure, the vaccine composition may be a vaccine composition in the form of a liposome formulation including a RSV antigen, CoPoP, and QS-21.

In an embodiment of the present disclosure, the vaccine composition may be a vaccine composition in the form of a liposome formulation including a RSV antigen, MLA, CoPoP, and QS-21.

Although the vaccine composition according to an embodiment of the present disclosure, comprising EcML and/or CoPoP may induce a strong immune response, optionally, the immune response may be further enhanced by adding an immune adjuvant. QS-21 promotes both humoral and cellular immune responses. Thus, if QS-21 is added to the vaccine composition, the vaccine composition may exhibit enhanced immunological efficacy. Applying the CoPoP technology to the liposome formulation including EcML and QS-21 as immune adjuvants, would make it possible to present a vaccine antigen of interest stably on the surface of the liposome to mimic virus as a VLP (virus-like particle) does. Further, the liposome formulation includes both the immune adjuvants and the vaccine antigen. Thus, the liposome formulation may maximize the vaccine efficacy.

The vaccine composition according to an embodiment of the present disclosure exhibits no or low risk of developing vaccine-associated enhanced respiratory disease (VAERD) compared to an antigen alone or a combination of the antigen and an immune adjuvant. VAERD refers to a vaccine-derived disease occurring from natural infection with RSV after vaccination against a respiratory infectious virus such as RSV due to immunogenicity derived therefrom.

In an embodiment of the present disclosure, the RSV antigen may be RSV F (fusion) glycoprotein E2 (RSVF-E2) antigen.

As used herein, the term “F glycoprotein” refers to a protein that mediates fusion of RSV and cell membranes for viral entry into the cell and fusion of membrances of infected cells and adjacent cells, which are essential in RSV infection, and may be used interchangeably with “F protein”, “fusion protein”, or the like.

In an embodiment of the present disclosure, the RSVF-E2 antigen may have an amino acid sequence of SEQ ID NO: 1.

RSVF-E2 is designed such that an ectodomain of the F protein of RSV is present in a stable prefusion form to have excellent immunogenicity and vaccine efficacy.

In an embodiment of the present disclosure, the vaccine composition may be a liposome formulation including EcML, which is MLA derived from E. coli, cholesterol, a phospholipid, and CoPoP.

As used herein, the term “EcML” refers to E. coli-derived monophosphoryl lipid A and may be produced by recombinant E. coli transformed to produce MLA (See Korean Patent No. 2019331). Specifically, EcML may be obtained without any chemical treatment, from recombinant E. coli KHSC0055 capable of producing and accumulating on cell membranes, lipid A, which is a component of LPS after corepolysaccharide and O-antigen, and 1-dephosphorylated-lipid A, without any influence on cell survival.

In an embodiment of the present disclosure, the antigen may include a polyhistidine tag. The polyhistidine tag binds to cobalt in CoPoP to effectively present the antigen on the surface of the liposome.

In an embodiment of the present disclosure, the polyhistidine tag may include 5 to 10 histidine residues. The polyhistidine tag may include, for example, 3 to 20, 3 to 16, 3 to 12, 3 to 8, 5 to 20, 5 to 16, 5 to 12, 5 to 8, 6 to 20, 6 to 16, 6 to 12, 6 to 10, or 6 to 8 histidine residues.

In an embodiment of the present disclosure, at least a portion of the polyhistidine tag is present in the hydrophobic portion of the monolayer or bilayer of the liposome, and at least one histidine residue of the polyhistidine tag forms a coordinate bond with cobalt of the CoPoP so that at least a portion of the RSV antigen is exposed to the exterior of the liposome.

In an embodiment of the present disclosure, the vaccine composition may further include an additional immune adjuvant. For example, the vaccine composition may further include an immune adjuvant, which may include but without being limited thereto, magnesium hydroxide, aluminum hydroxide, aluminum phosphate, and hydrated aluminum potassium sulfate (Alum).

In an embodiment of the present disclosure, the vaccine composition may further include an additional RSV antigen. The additional RSV antigen may have an epitope different from that of RSVF-E2, and thus enhance vaccine efficacy of the vaccine composition.

In an embodiment of the present disclosure, the RSVF-E2 antigen may be included in an amount of about 0.0001 wt % to about 10 wt %, about 0.0001 wt % to about 5 wt %, about 0.0001 wt % to about 1 wt %, about 0.0001 wt % to about 0.5 wt %, about 0.0001 wt % to about 0.1 wt %, about 0.0001 wt % to about 0.05 wt %, about 0.001 wt % to about 10 wt %, about 0.001 wt % to about 5 wt %, about 0.001 wt % to about 1 wt %, about 0.001 wt % to about 0.5 wt %, about 0.001 wt % to about 0.1 wt % or about 0.001 wt % to about 0.05 wt % based on a total weight of the vaccine composition, but is not limited thereto.

The vaccine composition according to an embodiment of the present disclosure may be administered to a subject to induce an immune response that protects the subject against a symptom or disease caused by a pathogen. The vaccine composition may be administered to prevent or treat (for example, to reduce or ameliorate) a symptom or disease caused by a pathogen by inhibiting replication of the pathogen after exposure of a subject to the pathogen. The vaccine composition may be administered as a therapeutic agent alone or in combination with other therapeutic agents sequentially or simultaneously therewith.

Another aspect of the present disclosure provides a method of preparing a vaccine composition comprising culturing host cells transfected with an expression vector including a gene encoding a RSVF-E2 (respiratory syncytial virus fusion glycoprotein E2) antigen and having a nucleotide sequence of SEQ ID NO: 2, and

obtaining the RSVF-E2 antigen having an amino acid sequence of SEQ ID NO: 1 from a culture of the host cells.

The vaccine composition may be a vaccine composition including a RSVF-E2 antigen having the amino acid sequence of SEQ ID NO: 1; and MLA, as described above.

As used herein, the term “expression vector” refers to a DNA construct containing a polynucleotide sequence encoding a protein of interest operably linked to regulatory sequences suitable for expressing the protein of interest in a suitable host. The regulatory sequences may include a promoter capable of initiating transcription, an operator sequence for regulating such transcription, a sequence encoding a suitable mRNA ribosome binding site, and a sequence regulating termination of transcription and translation. After a vector is transfected into a suitable host cell, the vector may be replicated or function independently of the host genome, or may be integrated into the genome itself.

In the method of preparing the vaccine composition, the vector used therefor is not particularly limited as long as it may be replicated in host cells, and any vector known in the art may be used. Examples of commonly used vectors include naturally occurring or recombinant plasmids, cosmids, viruses, and bacteriophages. For example, pWE15, M13, λMBL3, λMBL4, λIXII, λASHII, λAPII, λt10, λt11, Charon4A, and Charon21A may be used as phage vectors or cosmid vectors, and pDZ vectors, pBR vectors, pUC vectors, pBluescriptlI vectors, pGEM vectors, pTZ vectors, pCL vectors, and pET vectors may be used as plasmid vectors. Vectors that may be used in the method are not particularly limited, and any expression vectors known in the art may be used.

As used herein, the term “transfection” refers to a process of artificially making a genetic change by introducing an exogenous gene into a host cell so that the gene may be replicated as an episome or as part of the host genome by completion of chromosomal integration. Any transfection method may be used as the transfection method of the present disclosure, and the transfection may be readily conducted according to a method known in the art.

In addition, as used herein, the term “operably linked” may mean that a polynucleotide encoding a protein of interest is functionally linked to a promoter sequence that initiates and mediates transcription of the polynucleotide.

In an embodiment of the present disclosure, the expression vector may be constructed from pcDNA3.4 vector.

In an embodiment of the present disclosure, the RSVF-E2 antigen may include a polyhistidine tag at the C-terminal.

In an embodiment of the present disclosure, the host cell may be a CHO cell.

In an embodiment of the present disclosure, the host cell may be a host cell transformed to stably express a gene encoding the RSVF-E2 antigen.

As used herein, the term “stably express” means that a host cell permanently express a gene introduced thereinto by transformation. The “stably expressing” host cell may be used interchangeably with the “permanently expressing” host cell.

In the step of culturing the transfected host cells, type of a culture medium, culturing temperature, and culturing conditions may be determined as known in the art. The culture medium may include antibiotics. The antibiotics may be, for example, kanamycin, ampicillin, chloramphenicol, or a combination thereof.

As used herein, the term “culture” refers to a product obtained after culturing the transfected host cells according to a known microbial culturing method, and the culture may include a culture supernatant or cell lysate or homogenate and thus may be cell-free or not.

In the step of obtaining the RSVF-E2 antigen, the RSVF-E2 antigen may be obtained by a conventional method of isolating and purifying proteins as known in the art. For example, the RSVF-E2 antigen may be isolated and purified from the culture of the host cells by chromatography, but without being limited thereto. In an embodiment of the present disclosure, a recombinant RSVF-E2 antigen may be isolated and purified by collecting a supernatant of the culture of transfected host cells expressing the RSVF-E2 antigen, and subjecting the supernatant to affinity chromatography (e.g., using Ni Sepharose excel), anion exchange chromatography (e.g., using Q Sepharose XL), cation exchange chromatography (e.g., using Capto S ImpAct), and size exclusion chromatography (e.g., using Superdex 75).

In an embodiment of the present disclosure, the obtaining of the RSVF-E2 antigen may include purifying a culture of the host cells by affinity chromatography to obtain a first eluate, purifying the first eluate by anion exchange chromatography to obtain a second eluate, purifying the second eluate by cation exchange chromatography to obtain a third eluate, and purifying the third eluate by tangential flow filtration (TTF) to obtain the RSVF-E2 antigen.

In an embodiment of the present disclosure, the affinity chromatography may be performed by using Ni Sepharose® excel, the anion exchange chromatography may be performed by using Q Sepharose XL, the cation exchange chromatography may be performed by using Capto S ImpAct, and the TTF may be performed by using 50 k Da cut-off membrane.

In an embodiment of the present disclosure, the vaccine composition may be a liposome formulation, and the method may further include preparing a MLA-containing liposome formulation by adding MLA to a composition for preparing a liposome, including a phospholipid and cholesterol.

In an embodiment of the present disclosure, the vaccine composition may be a liposome formulation, and the method may further include preparing a CoPoP-containing liposome formulation by adding CoPoP to a composition for preparing a liposome, including a phospholipid and cholesterol.

In an embodiment of the present disclosure, the method may further include mixing the RSVF-E2 antigen with the MLA-containing liposome formulation, and mixing saponin with the liposome formulation mixed with the RSVF-E2 antigen.

In an embodiment of the present disclosure, the preparing of the liposome formulation may further include adding CoPoP to the composition for preparing a liposome to prepare a liposome formulation including MLA and CoPoP.

In an embodiment of the present disclosure, the method may further include mixing the RSVF-E2 antigen with the CoPoP-containing liposome formulation, and mixing a saponin with the liposome mixed with the RSVF-E2 antigen.

In an embodiment of the present disclosure, the preparing of the liposome formulation may further include adding MLA to the composition for preparing a liposome to prepare a liposome formulation including MLA and CoPoP.

In an embodiment of the present disclosure, the method may further include mixing the RSVF-E2 antigen with the MLA and CoPoP containing liposome formulation to make the RSVF-E2 antigen bind to the liposome formulation, and mixing saponin with the liposome with the RSVF-E2 antigen bound thereto.

In an embodiment of the present disclosure, the saponin may be QS-21.

In an embodiment of the present disclosure, the method may further include mixing the RSVF-E2 antigen with the MLA and CoPoP containing liposome formulation to make the RSVF-E2 antigen bind to the liposome formulation, and mixing QS-21 with the liposome with the RSVF-E2 antigen bound thereto.

Another aspect of the present disclosure provides a method of preventing or treating a disease caused by RSV infection, the method including administering the vaccine composition to a subject that may be infected with RSV or has the disease caused by RSV infection.

The vaccine composition is as described above.

The vaccine composition may be administered to the subject by any know method in the art. For example, the vaccine composition may be directly administered to the subject via any route such as an intravenous, intramuscular, or oral route.

The vaccine composition may be administered in a pharmaceutically effective amount. As used herein, the term “pharmaceutically effective amount” refers to an amount sufficient to lead to vaccine efficacy without causing side effects or serious or excessive immune responses, and the effective amount may vary according to various factors such as the disorder to be treated, severity of the disorder, activity of a specific treatment to be used, route of administration, excretion rate of the vaccine composition, duration of treatment, drug used in combination or concurrently with the vaccine composition, age, weight, gender, diet, and general health of a subject, and other factors known in the pharmaceutical and medical art. Various general considerations in determining the pharmaceutically effective amount are known to those skilled in the art and are described, for example, in documents [Gilman et al., eds., Goodman And Gilman's: The Pharmacological Bases of Therapeutics, 8th ed., Pergamon Press, 1990] and [Remington's Pharmaceutical Sciences, 17th ed., Mack Publishing Co., Easton, Pa., 1990]. The vaccine composition may be administered to non-human animals in the same dosage per kg as that to humans or in a dosage converted from the above-described dosage based on, for example, a volume ratio (e.g., average value) between organs (heart, etc.) of the human and animal subjects.

The subject may be mammal, such as, human, mouse, rat, cow, horse, pig, dog, sheep, goat, cat, or ape.

As used herein, the term “prevention” refers to any activity that suppresses or delays an onset of a disease caused by RSV infection by administering the vaccine composition of the present disclosure.

As used herein, the term “treatment” refers to any activity that ameliorates or beneficially changes the symptoms of a disease already caused by RSV infection by administering the vaccine composition of the present disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic diagram illustrating development of a CHO cell line stably expressing an RSV antigen according to an embodiment of the present disclosure.

FIG. 2 is a map of a recombinant vector for expressing RSVF-E2, an RSV antigen according to an embodiment of the present disclosure.

FIG. 3 is a schematic diagram of a process of culturing a RSV antigen-producing cell line according to an embodiment of the present disclosure.

FIG. 4 shows results of western blotting of cultures of a cell line permanently expressing RSVF-E2 antigen according to an embodiment of the present disclosure on Day 8 (8D), Day 10 (10D), and Day 12 (12D).

FIG. 5 shows results of SDS-PAGE and western blotting of purification steps using a cell line permanently expressing RSVF-E2 antigen according to an embodiment of the present disclosure.

FIG. 6 shows SEC-HPLC based purity analysis of RSVF-E2 antigens produced and purified according to an embodiment of the present disclosure.

FIG. 7 shows measurement results of antigen-specific serum antibody titer of a vaccine composition according to an embodiment of the present disclosure.

FIG. 8 shows neutralizing antibody titers of a vaccine composition according to an embodiment of the present disclosure for RSV A2.

FIG. 9 shows neutralizing antibody titers of a vaccine composition according to an embodiment of the present disclosure for RSV 18537.

FIG. 10 shows measurement results of RSV-specific cell-mediated immunity of a vaccine composition according to an embodiment of the present disclosure.

FIG. 11 shows protective activity of a vaccine composition according to an embodiment of the present disclosure against RSV challenge.

FIG. 12 shows the probability of causing a vaccine-associated enhanced respiratory disease (VAERD) by a vaccine composition according to an embodiment of the present disclosure.

### DETAILED DESCRIPTION

Reference will now be made in detail to embodiments, examples of which are illustrated in the accompanying drawings, wherein like reference numerals refer to like elements throughout. In this regard, the embodiments may have different forms and should not be construed as being limited to the descriptions set forth herein. Accordingly, the embodiments are merely described below, by referring to the figures, to explain aspects of the description. As used herein, the term “and/or” includes any and all combinations of one or more of the associated listed items. Expressions such as “at least one of,” when preceding a list of elements, modify the entire list of elements and do not modify the individual elements of the list.

Hereinafter, the present disclosure will be described in more detail with reference to the following examples. However, these examples are for illustrative purposes only, and the scope of the disclosure is not limited by these examples.

### Example 1. Preparation of Monophosphoryl Lipid A

1.1. Culture of Monophosphoryl Lipid A-Producing Strain

1.1.1. Preparation of Monophosphoryl Lipid A-Producing Strain and Culture Medium

Escherichia coli (E. coli) KHSC0055 strain disclosed in Korean Patent No. 10-2019331 was used as a strain producing monophosphoryl lipid A. The strain was cultured in a 30 L-fermenter, and culture media for seed culture and main culture were separately prepared. The medium for seed culture was prepared by dissolving 16.0 g/L of phytone peptone, 10 g/L of yeast extract, and 5 g/L of NaCl in purified water such as D.W., titrating the solution to a pH of 7.2±0.2, and autoclaving the solution. The medium for main culture was prepared by sterilizing a solution including 3.5 g/L of phytone peptone, 21.0 g/L of yeast extract, 6.0 g/L of KH₂PO₄, 5.0 g/L of K₂HPO₄, and 5.0 g/L of NH₄Cl in a fermenter, and 40.0 g/L of glucose and 10.0 g/L of MgSO₄·7H₂O were separately prepared and sterilized for aseptical addition to the fermentor during the culture, as feed.

1.1.2. Seed Culture

For primary seed culture, 200 ml of the sterile seed culture medium was added to a 1 L-Erlenmeyer flask. A seed vial of the KHSC0055 strain was thawed, added to the flask, and incubated in a shaking incubator at 30±1° C. for about 18 to about 22 hours.

For secondary seed culture, 600 ml of the sterile seed culture medium was added to each of six 2 L-Erlenmeyer flasks, and 35 ml of a culture broth of the primary seed culture was inoculated thereinto. The culture was incubated in a shaking incubator at 30±1° C. for about 7 to about 12 hours.

1.1.3. Main Culture

The main culture was performed in a 30 L-fermenter with an initial working volume of 18 L. The fermenter was filled with 18 L of the main culture medium and sterilized with high pressure steam, and a glucose solution was separately prepared and sterilized and added to the fermenter aseptically during the culture. The concentration of glucose in the culture broth was adjusted to be 1 g/L or less. After sterilization, the pH of the medium was maintained at a pH of 7.2±0.2 using NH₄OH. A culture broth of the second seed culture was inoculated into the fermenter, and incubated at 30±1° C. with 3.0 Lpm of aeration and 50% of dissolved oxygen (DO) while stirring at about 250 rpm to about 400 rpm. A growth stage of the culture was monitored by measuring absorbance at 600 nm.

The culture broth was harvested when the E. coli reached the death phase following the exponential phase and the stationary phase. The culture broth was centrifuged or filtered by tangential flow filtration, washed with PBS, and then frozen.

1.2. Lipid Extraction

Lipids were extracted according to the Bligh-Dyer system. The culture broth collected in Section 1.1 was centrifuged at room temperature for about 20 minutes to obtain only E. coli. The obtained E. coli was suspended in purified water and a mixture of E. coli suspension:methanol:chloroform at the ratio of 5:12.5:6.25 (v/v) was incubated at room temperature for 1 hour while shaking. The incubated mixture was centrifuged at room temperature for about 30 minutes to collect a supernatant. Purified water and chloroform were each added to the obtained supernatant at a rate of 6.25 (v/v), and thoroughly mixed, followed by centrifugation at room temperature for 20 minutes. An organic solvent layer was separated from the centrifuged mixture, dried in a nitrogen dryer to obtain lipids, and the lipids were frozen for storage.

1.3. Lipid Crystallization

Phospholipid crystallization was performed to remove pigments derived from the culture broth and impurities derived from E. coli cell membranes and to adjust levels of homologs of EcML. Total lipids extracted from the strain as in Section 1.2 above were dissolved in chloroform to obtain a total lipid solution. The total lipid solution was added dropwise to methanol in a volume 10 to 100 times a volume of chloroform in which the total lipids from the stain were dissolved, followed by stirring at room temperature for 1 hour or more and stirring under refrigeration for 1 hour or more to proceed crystallization reaction. The obtained crystals of the total lipids were recovered by Nutsche filtration.

1.4. Purification of Monophosphoryl Lipid A

Two-step column purification, ion-exchange chromatography, and reverse phase chromatography were performed to purify monophosphoryl lipid A from the total lipids obtained in Section 1.3.

To isolate 1-dephosphorylated-lipid A from the mixture of lipid A and 1-dephosphorylated-lipid A, purification was performed by using an ammonium acetate gradient. Any salts other than the ammonium acetate may also be used. After conducting a washing process to remove impurities at a low salt concentration, EcML was eluted at a medium salt concentration. Subsequently, a resin was regenerated by eluting lipid A at a high salt concentration. Based on the result of the TLC (Thin Layer Chromatography) analysis, fractions in which only 1-dephosphorylated-lipid A were eluted were selected and pooled in a separatory funnel. To obtain an organic solvent layer in which only 1-dephosphorylated-lipid A was dissolved, washing and layer separation were performed by using NaCl. The separatory funnel was well shaken to uniformly mix all solutions and then left until the mixture was separated into two layers. Then, when a single phase in which an aqueous solution layer was mixed with an organic solvent layer containing dissolved monophosphoryl lipid A was separated into to two phases, the organic solvent layer and the aqueous solution layer, only the organic solvent layer was isolated to obtain monophosphoryl lipid A.

1-dephosphorylated-lipid A was obtained by removing lipid A by ion-exchange chromatography and was named crude 1-dephosphorylated-lipid A (crude EcML).

After obtaining 1-dephosphorylated-lipid A (crude EcML) by ion-exchange chromatography, reverse phase chromatography was conducted to remove remains of E. coli-derived phospholipids and adjust levels of homologs of monophosphoryl lipid A.

SMT bulk C8 resin (Separation Methods Technologies, Inc.) was used as a resin for the separation and purification. The resin may be a C18 or C4 resin instead of the C8 resin. The purification was conducted by using differences in polarity of the solvents. To this end, chloroform, methanol, and purified water were mixed in a certain ratio and a step gradient was applied thereto from relatively polar conditions to nonpolar conditions.

After purification, fractions in which EcML was eluted were selected by thin layer chromatography (TLC) and pooled. A volume of the pooled solution (hereinafter, referred to as C8 eluate) was measured. Layer separation was conducted three times by adding to a C8 eluate the corresponding volume of chloroform, 1% NaCl for preventing leaching of ammonium acetate, and purified water. A lower layer (C8-eluate, EcML) was collected and the volume thereof was determined. A sample thereof was analyzed to determine the amount of EcML by gas chromatography (GC). A finally obtained EcML was dried and stored under refrigeration.

### Example 2: Design and Production of Respiratory Syncytial Virus (RSV) Fusion (F) Glycoprotein Antigen

2.1. Design of RSV F Glycoprotein Antigen

RSV includes a fusion (F) glycoprotein, which is a transmembrane glycoprotein and plays a critical role in binding and fusion to a host cell and infection. A RSVF-E2 antigen was designed such that the ectodomain of the F protein was present in a stable prefusion form. The designed RSVF-E2 antigen is represented by an amino acid sequence of SEQ ID NO: 1.

2.2. Construction of Cell Line Producing RSVF-E2 Antigen

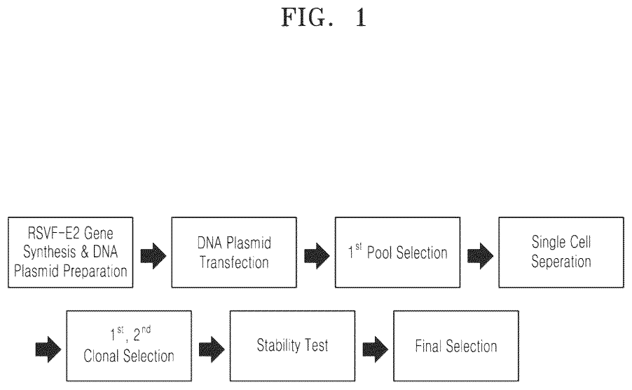

A gene encoding the RSVF-E2 obtained in Section 2.1 was synthesized, and a cell line stably and highly expressing the recombinant DNA was selected to obtain a CHO cell-based RSVF-E2 antigen-producing cell line. FIG. 1 shows a process of selecting a cell line.

Chinese Hamster Ovary (CHO) cells, which produce high-quality proteins by post-translational modification and are easy to insert target proteins, were chosen as a parent cell system for production of the RSVF-E2 antigen. Antigen-producing cells were developed by using ExpiCHO-S cells (ThermoFisher), a type of CHO cells modified and developed to increase expression of proteins.

Construction of RSVF-E2 Antigen Expression Vector

An expression vector was constructed by using a nucleotide sequence encoding RSVF-E2 as designed in 2.1.

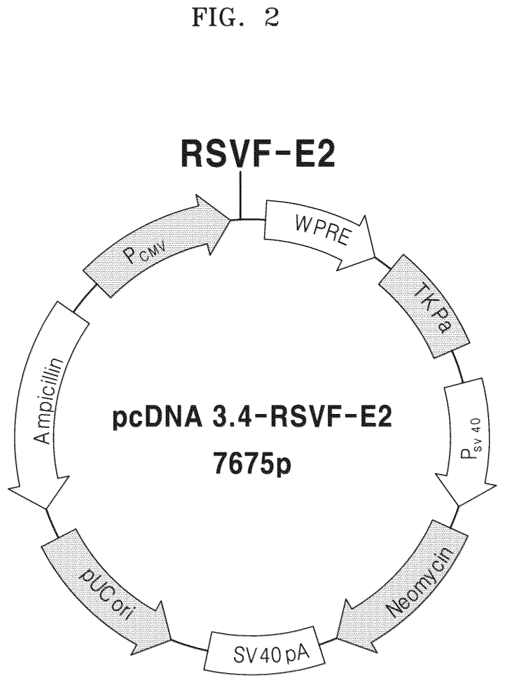

As a base vector, pcDNA3.4 was selected because its high expression level and high preservation level in CHO cell lines, and small size were advantageous for the development of the expression vector. The pcDNA3.4 used in the development of antigen-producing cell lines was purchased from ThermoFisher. The vector can be used for development of cell lines transiently or permanently expressing proteins. The RSVF-E2 antigen expression vector includes elements required for expression in animal cells to produce the antigen, such as a Kozak sequence, a promoter, an enhancer, selection markers, and origin of replication. FIG. 2 shows a map of the RSVF-E2 antigen expression vector, pcDNA 3.4-RSVF-E2.

A nucleotide sequence encoding RSVF-E2 was synthesized by Integrated DNA Technologies (IDT) via codon optimization for CHO cells and inserted into the vector by TA cloning. Specifically, the RSVF-E2 gene of SEQ ID NO: 2 was amplified in a form with a single deoxyadenosine (A) added to the 3′-terminal by PCR using a Taq polymerase, and ligated into pcDNA™ 3.4-TOPO® vector having a single 3′ deoxythymidine (T). In order to select complete clones formed by binding between deoxyadenosine (A) and deoxythymidine (T), E. coli TOP10 was transformed with the DNA, and colony PCR was performed to select candidates, nucleotide sequence analysis was conducted for confirmation and finally the vector was obtained. By confirming that the amino acid sequence encoded by the nucleotide sequence of the constructed vector was 100% identical to the amino acid sequence of the RSVF-E2 antigen, the RSVF-E2 antigen expression vector, pcDNA 3.4-RSVF-E2 was obtained.

A RSVF-E2 antigen-producing cell line was constructed by transfecting the pcDNA 3.4-RSVF-E2 into ExpiCHO-S cells (ThermoFisher).

2.3. Culture of RSVF-E2 Antigen-Producing Cell Line

The RSVF-E2 antigen-producing cell line obtained in Section 2.2 was cultured to produce RSVF-E2 antigen.

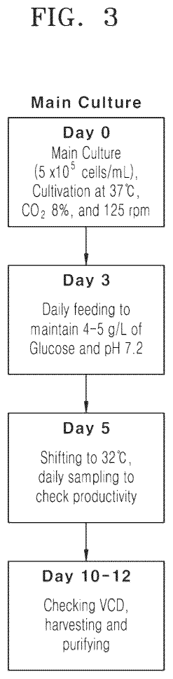

The RSVF-E2 antigen-producing cell line was cultured by seed culture and main culture to produce RSVF-E2 antigen. The main culture process is shown in FIG. 3.

Specifically, the seed culture was performed in a seed culture medium prepared by adding GlutaMAX Supplement to an ExpiCHO Stable Production AGT medium, followed by sterile filtration. The main culture was performed in a main culture medium prepared by adding a nicotinamide solution, a Myo-inositol solution, an aurintricarboxylic acid (ATA) solution, GlutaMAX Supplement, and Pluronic F-68 15 to an ExpiCHO Stable Production AGT Medium, followed by sterile filtration. For the seed culture, the seed culture medium was divided into an Erlenmeyer flask, 1 ml of a working cell bank (WCB) vial was inoculated thereinto, followed by incubation at a temperature of 36.5±0.5° C., at a stirring rate of 150±10 rpm, and under CO₂ conditions of 8.0±1.0%. When a final viable cell density (VCD) and cell viability satisfied preset criteria, the seed culture was inoculated onto the main culture medium, and 2× Feeding (culture medium of 2× concentration) was fed in 2% of an initial volume from Day 3 of the main culture, and glucose was supplied thereto when a glucose concentration was 4.0 g/L or less. When a viable cell density (VCD) was 1.5×10⁷ cells/mL or more during the main culture or on Day 5, a culture temperature was changed from 36.5±0.5° C. to 32° C. The main culture was terminated after 10 days or when the cell viability decreased to 70% or lower. Upon termination of the main culture, the culture was centrifuged at 5000 rpm, at 4° C., for 20 minutes to separate cells by using a high-speed centrifuge, and a supernatant was recovered and used for purification of RSVF-E2 antigen.

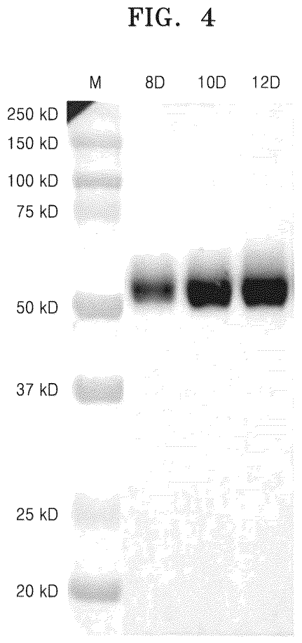

On Day 8, Day 10, and Day 12 of the culture, 1 ml of a culture broth was sampled and analyzed by Western blotting, and RSVF-E2 bands at around 50 Kda were identified. Specifically, 10 μl of a supernatant from which the cells were removed was loaded into each well and analyzed by western blotting by using Anti-F(RSV)D25, a monoclonal antibody from Cambridge Bio. Results of western blotting are shown in FIG. 4.

2.4. Purification of RSVF-E2 Antigen

The supernatant from the culture broth obtained in Section 2.3 was subjected to a three-step column process and a 50 kDa ultrafiltration/dialysis (UF/D) process to effectively remove host cell-derived proteins (impurities) to produce high-quality RSVF-E2 antigen.

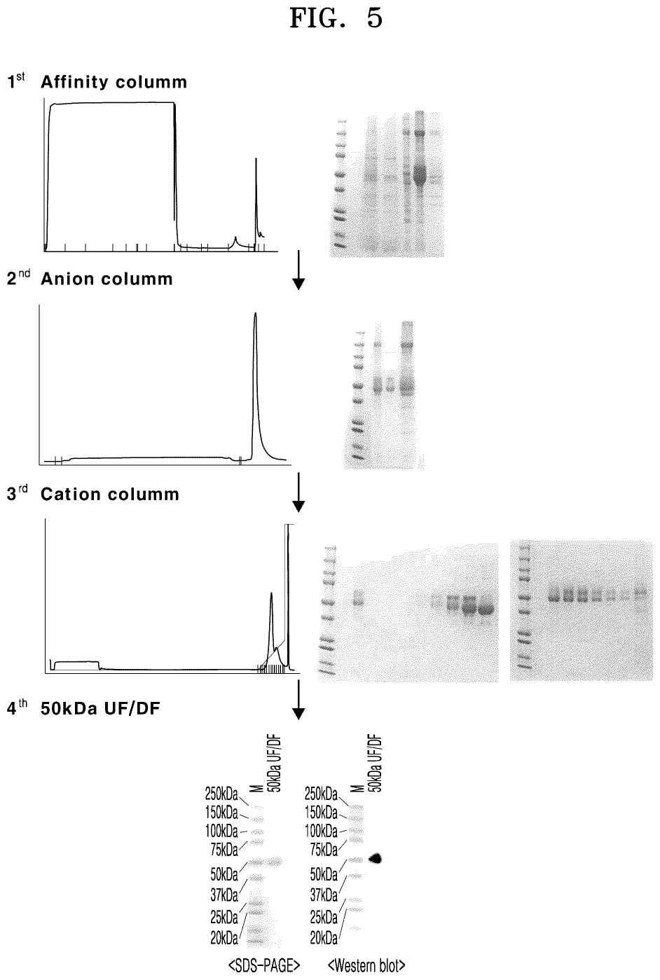

A first column process was affinity chromatography (Ni Sepharose excel). Impurities contained in the culture broth were removed in a flow through and during a washing process, and the RSVF-E2 antigen having a tag consisting of 6 histidine residues was purified after being bound to nickel contained in a resin and eluted. Specifically, impurities having a low binding affinity to nickel were removed by flowing a washing buffer with optimized conditions (30 mM NaPO₄, 10 mM imidazole, pH 7.3) through the column, and the antigen was physiochemically eluted from the resin by flowing an elution buffer containing an optimized concentration of imidazole (30 mM NaPO₄, 500 mM imidazole, pH 7.3) to obtain a first eluate. A second column process was anion exchange chromatography (Q Sepharose XL) in which the resin in the column is positively charged, and thus, a negatively charged protein binds thereto. By anion exchange chromatography (Q Sepharose XL), the RSVF-E2 antigen in the first eluate, having a (+) charge under pH 7.3 buffer conditions due to an inherent isoelectric point thereof was obtained in the flow through, while protein impurities bound to the resin, thereby further removing impurities from the first eluate to obtain a second eluate. A third column process was cation exchange chromatography (Capto S ImpAct) in which the resin in the column is negatively charged and thus, a positively charged protein binds thereto. The RSVF-E2 antigen has a (+) charge under the pH 7.3 buffer conditions due to the inherent isoelectric point. The second eluate obtained from the second column process was passed through a Capto S ImpAct column to remove unbound proteins and impurities, followed by a washing process to remove protein impurities with a low binding affinity, except for the RSVF-E2 antigen, depending on a salt concentrations. Subsequently, the RSVF-E2 antigen was eluted by gradually increasing the salt concentration to obtain a third eluate. The Tangential flow filtration (TFF) system was applied to effectively remove host cell-derived proteins from the RSVF-E2 antigen in the third eluate. The third eluate was concentrated to 1.5 mg/mL by using a 50 kDa cut-off membrane, and diafiltration (DF) was performed with a 1×PBS buffer in a volume 30 times a volume of a retentate. Cell-derived proteins that were not removed in the column processes and having a molecular weight lower than 50 kDa were removed to obtain high-purity antigen, satisfying a host-derived protein standard (100 ppm). FIG. 5 shows a schematic diagram of a method of purifying the RSVF-E2 antigen with results of SDS-PAGE and western blotting of samples of each process.

Levels of host cell proteins (HCPs) derived from host cells were measured in the RSVF-E2 antigens obtained by the purification method according to the present embodiment including the three optimized column processes and the 50 kDa ultrafiltration/dialysis (UF/D) process and those obtained by a conventional purification method consisting of affinity chromatography (Ni Sepharose excel), anion exchange chromatography (Q Sepharose XL), cation exchange chromatography (Capto S ImpAct), and size exclusion chromatography (Superdex 75) and compared therebetween. The level of HCPs in the RSVF-E2 antigen purified by the method according to the present embodiment was significantly lower than that of the antigen purified by the conventional method. The Analysis results are shown in Table 1.

TABLE 1

| | |
|---|---|
| Conventional purification | Purification method of present |
| method | embodiment |

| Batch No. | HCP (ppm) | Batch No. | HCP (ppm) |
|---|---|---|---|
| RSVF-E2-015S | 407 | RSVF-E2-021S | 26.2 |
| RSVF-E2-017S | 108.3 | RSVF-P-ENG | 29.9 |
| RSVF-E2-018S | 484.8 | RSVF-P2301 | 32.8 |

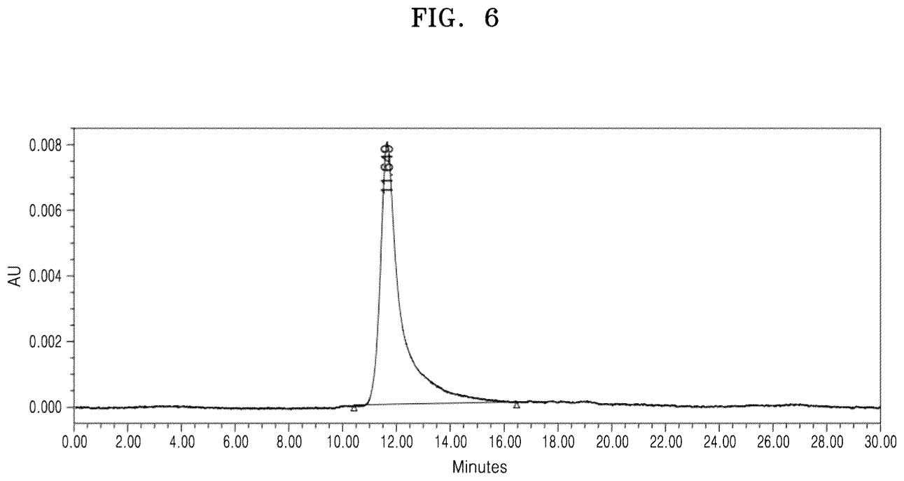

To analyze quality of the RSVF-E2 antigen, a relative content (%) of main peak area of an undiluted solution of the antigen was measured by SEC-HPLC. The mobile phase was PBS and a flow rate was set at 0.6 mL per minute. A column was mounted and 50 μl of an undiluted antigen solution was injected into the column for analysis at 280 nm using an UV detector. The main peak area was calculated as the relative content to the area of all peaks and it was found that purity was about 99% or more based on the analysis results. Analysis results are shown in FIG. 6.

### Example 3. Preparation of Liposome Formulation

A liposome formulation of a vaccine was prepared to efficiently deliver an antigen such as the RSVF-E2 antigen and induce an immune response thereto.

A liposome formulation was prepared, to which an immune adjuvant, EcML and/or CoPoP, which enables an antigen to bind to the surface of a liposome, were added. Abbreviations and compositions of liposome formulations are shown in Table 2 below.

TABLE 2

| | |
|---|---|
| CLS | Liposome consisting of CoPoP, DOPC, and cholesterol |
| ELS | Liposome consisting of EcML, DOPC, and cholesterol |
| ECLS | Liposome consisting of EcML, CoPoP, DOPC, and cholesterol |
| PCLS | Liposome consisting of PHAD*, CoPoP, DOPC, and cholesterol |
| | *PHAD(3D-(6-acyl) PHAD ®): MPLA produced by |
| | synthesis by Avanti |
| Q | QS21, saponin-based immune adjuvant |
| CLSQ | Liposome prepared by adding QS21 to CLS |
| ELSQ | Liposome prepared by adding QS21 to ELS |
| ECLSQ | Liposome prepared by adding QS21 to ECLS |
| PCLSQ | Liposome prepared by adding QS21 to PCLS |

1,2-dioleoyl-sn-glycero-3-phosphocholine (DOPC), a phospholipid as an auxiliary lipid, and cholesterol are added to construct a liposome formulation, and these components are essential to maintain physical strength and stability of the liposome formulation.

Using chloroform as a solvent, 25 mg/mL of the DOPC solution, 10 mg/mL of cholesterol, 1 mg/mL of CoPoP (U.S. Pat. No. 10,272,160 B2), 1 mg/mL of QS-21, and 1 mg/mL of EcML purified in Example 1 were prepared respectively. 0.8 mL of the DOPC solution, 0.5 mL of cholesterol, and 0.5 mL to 1 mL of EcML were added to a sterilized glass vial and mixed to prepare an ELS mixture. 0.8 mL of the DOPC solution, 0.5 mL of cholesterol, and 1 mL of CoPoP were added to a sterilized glass vial and mixed to prepare a CLS mixture. In addition, 1 mL of CoPoP was added to the ELS mixture and mixed to prepare an ECLS mixture.

Each of the mixtures for the formulations was formed to thin lipid films by completely evaporating the solvent by using a rotary vacuum evaporator, and PBS, pH 7.2 was added thereto, followed by re-hydration in a sonicator set at 60° C. for 1 to 2 hours for first homogenization. Second homogenization was performed by using an extruder or a microfluidizer so that sizes of final liposome formulations ELS, CLS, and ECLS were in the range of 50 to 150 nm, respectively. In this regard, the size of the liposome formulation was analyzed by a device using dynamic light scattering (DLS).

ELSQ, CLSQ, and ECLSQ were prepared respectively by mixing each of the prepared ELS, CLS, and ECLS with QS-21. Sterile filtration was performed with a 0.2 μm PES syringe filter in a sterile field, and the amounts of EcML, CoPoP, DOPC, and cholesterol in the liposome formulations were quantified by using a high performance liquid chromatography-charged aerosol detector (HPLC-CAD), and the amounts of QS21 in the prepared liposome formulations were quantified by a high performance liquid chromatography-UV-vis detector (HPLC-UVD).

### Example 4. Binding of Liposome Formulation to Antigen

The RSVF-E1 antigen (SEQ ID NO: 3) derived from F protein of RSV and the RSVF-E2 antigen (SEQ ID NO: 1) designed in Example 1 were respectively bound to each of the liposome formulations CLS, ECLS, and ECLSQ prepared in Example 3. Specifically, each of the RSVF-E1 and RSVF-E2 antigens was mixed with liposomes and left at room temperature for 2 hours so that the antigens bind to CoPoP in each of CLS, ECLS, and ECLSQ Iiposomes. Then, the resulting products were diluted with PBS and stored under refrigeration. In the case of the CoPoP-free ELS and ELSQ Iiposomes, each of the RSVF-E1 and RSVF-E2 antigens was simply mixed with the liposomes.

### Example 5. Identification of Efficacy and Side Effect of RSV Vaccine Composition

In order to identify efficacy and side effects of the combination of immune adjuvants for the RSV vaccine composition, alum, the liposome formulations CLS, ELS, ECLS, and PCLS prepared in Example 3, and QS21 were used as immune adjuvants.

5.1. Test Group Setup and Administration of Vaccine Composition

A total of 21 test groups were prepared. Each test group consists of five 6-week-old BALB/cAnNCrljOri (female) mice.

Test group 1 was administered with 1×PBS as a negative control, Test groups 2 and 12 were administered with antigen alone (RSVF-E1 and RSVF-E2), Test groups 3 and 13 were administered with the formulations in which alum was mixed with each antigen (RSVF-E1/Alum and RSVF-E2/Alum), Test groups 4 and 14 were administered with the formulations in which CLS was mixed with each antigen (RSVF-E1/CLS and RSVF-E2/CLS), Test groups 5 and 15 were administered with formulations in which ELS was mixed with each antigen (RSVF-E1/ELS and RSVF-E2/ELS), Test groups 6 and 16 were administered with formulations in which ECLS was mixed with each antigen (RSVF-E1/ECLS and RSVF-E2/ECLS), Test groups 7 and 17 were administered with formulations in which PCLS was mixed with each antigen (RSVF-E1/PCLS and RSVF-E2/PCLS), Test groups 8 and 18 were administered with formulations in which CLSQ was mixed with each antigen (RSVF-E1/CLSQ and RSVF-E2/CLSQ), Test groups 9 and 19 were administered with formulations in which ELSQ was mixed with each antigen (RSVF-E1/ELSQ and RSVF-E2/ELSQ), Test groups 10 and 20 were administered with formulations in which ECLSQ was mixed with each antigen (RSVF-E1/ECLSQ andRSVF-E2/ECLSQ), and Test groups 11 and 21 were administered with formulations in which PCLSQ was mixed with each antigen (RSVF-E1/PCLSQ and RSVF-E2/PCLSQ), as vaccine compositions.

The vaccine compositions used in the test groups administered with CLSQ, ELSQ, ECLSQ, and PCLSQ were prepared by mixing QS-21 with RSVF-E2-bound CLS, RSVF-E2-bound ECLS, RSVF-E2-bound PCLS, and ELS liposomes mixed with RSVF-E2, respectively, in the same amount (5 μg) as that of monophosphoryl lipid A contained in each liposome.

The prepared vaccine composition for each of the test groups was intramuscularly administered to thighs of the mice with a volume of 50 μl twice at a 3-week interval. Table 3 shows formulation, antigen, and administration route of test groups.

TABLE 3

| | | | No. of | Administration | | | | |
|---|---|---|---|---|---|---|---|---|
| | | | animals | volume | | | | |
| Test | | | (head) | (μl) | Dose (μg) | | | Administration |
| group | Formulation | Antigen | Ag | | | MLA | QS-21 | route |
| 1 | PBS | — | 5 | 50 | — | — | — | Intramuscular |
| | | | | | | | | injection |
| 2 | Ag | RSVF-E1 | 5 | 50 | 5 | — | — | Intramuscular |
| | | | | | | | | injection |
| 3 | alum | RSVF-E1 | 5 | 50 | 5 | — | 50 (alum) | Intramuscular |
| | | | | | | | | injection |
| 4 | CLS | RSVF-E1 | 5 | 50 | 5 | 5 | — | Intramuscular |
| | | | | | | | | injection |
| 5 | ELS | RSVF-E1 | 5 | 50 | 5 | 5 | — | Intramuscular |
| | | | | | | | | injection |
| 6 | ECLS | RSVF-E1 | 5 | 50 | 5 | 5 | — | Intramuscular |
| | | | | | | | | injection |
| 7 | PCLS | RSVF-E1 | 5 | 50 | 5 | 5 | — | Intramuscular |
| | | | | | | | | injection |
| 8 | CLSQ | RSVF-E1 | 5 | 50 | 5 | 5 | 5 | Intramuscular |
| | | | | | | | | injection |
| 9 | ELSQ | RSVF-E1 | 5 | 50 | 5 | 5 | 5 | Intramuscular |
| | | | | | | | | injection |
| 10 | ECLSQ | RSVF-E1 | 5 | 50 | 5 | 5 | 5 | Intramuscular |
| | | | | | | | | injection |
| 11 | PCLSQ | RSVF-E1 | 5 | 50 | 5 | 5 | 5 | Intramuscular |
| | | | | | | | | injection |
| 12 | Ag | RSVF-E2 | 5 | 50 | 5 | — | — | Intramuscular |
| | | | | | | | | injection |
| 13 | alum | RSVF-E2 | 5 | 50 | 5 | — | 50 (alum) | Intramuscular |
| | | | | | | | | injection |
| 14 | CLS | RSVF-E2 | 5 | 50 | 5 | 5 | — | Intramuscular |
| | | | | | | | | injection |
| 15 | ELS | RSVF-E2 | 5 | 50 | 5 | 5 | — | Intramuscular |
| | | | | | | | | injection |
| 16 | ECLS | RSVF-E2 | 5 | 50 | 5 | 5 | — | Intramuscular |
| | | | | | | | | injection |
| 17 | PCLS | RSVF-E2 | 5 | 50 | 5 | 5 | — | Intramuscular |
| | | | | | | | | injection |
| 18 | CLSQ | RSVF-E2 | 5 | 50 | 5 | 5 | 5 | Intramuscular |
| | | | | | | | | injection |
| 19 | ELSQ | RSVF-E2 | 5 | 50 | 5 | 5 | 5 | Intramuscular |
| | | | | | | | | injection |
| 20 | ECLSQ | RSVF-E2 | 5 | 50 | 5 | 5 | 5 | Intramuscular |
| | | | | | | | | injection |
| 21 | PCLSQ | RSVF-E2 | 5 | 50 | 5 | 5 | 5 | Intramuscular |
| | | | | | | | | injection |

5.2. Measurement of Antigen-Specific Serum Antibody Titer

Enzyme-linked immunosorbent assay (ELISA) was used to measure RSV antigen-specific antibody titer in serum of mice after immunization. Each of the antigens (RSVF-E1 and RSVF-E2) diluted with PBS was aliquoted into each well of a 96-well plate at a density of 1 μg/mL, sealed, allowed to stand at 4° C. for one day, and washed three times with PBS including 0.05% Tween 20. 100 μl of PBS including 2% skim milk and 0.05% Tween 20 was aliquoted into each well of the 96-well plate, followed by incubation for 1 hour at 37° C. to prevent non-specific reaction of the antibody. Mouse serum samples obtained before immunization, after first immunization, and after second immunization were serially diluted (2-fold dilution) by using PBS including 2% skim milk and 0.05% Tween 20, and then 100 μl of each of the dilutions was aliquoted into each well of a 96-well plate bound to each antigen and sealed and incubated for 1 hour at 37° C. Thereafter, the plate was washed three times with PBS including 0.05% Tween 20, and anti-mouse IgG-HRP was diluted 1:5000 with PBS including 1% skim milk and 0.05% Tween 20. 100 μl of the dilutions was added to each well, sealed and incubated in a light-shielded state for 1 hour at 37° C. After washing the plate 3 times with PBS containing 0.05% Tween 20, 100 μl of TBM substrate was added to each well for color development by horseradish peroxidase (HRP), and after 10 minutes of reaction, 100 μl of 0.5 M H₂SO₄ was added to each well to stop the reaction. After the reaction was completed, absorbance of the plate was measured at 450 nm to determine the final antibody titer of each test group.

A cut-off value was calculated to set a criterion for classifying a test result as positive and negative based on the measurement results of pre-immunization mouse serum obtained by using the above-described test method. The cut-off value was calculated by Equation 1 below.

Cut-off value=average absorbance of pre-immunization mouse serum+2×standard deviation of absorbance of pre-immunization mouse serum. [Equation 1]

For the antibody titer calculated with mouse serum obtained after the first and second immunizations, the dilution factor corresponding to the measured absorbance higher than each cut-off value was determined as the final antibody titer. For example, when a cut-off value is 0.143, absorbance at 2¹⁴-fold dilution is 0.167, and absorbance at 2¹⁵-fold dilution is 0.124, 2¹⁴ becomes the final antibody titer of IgG. From the results calculated by the above method, an average and standard deviation for 5 mice per test group were calculated and converted into log values.

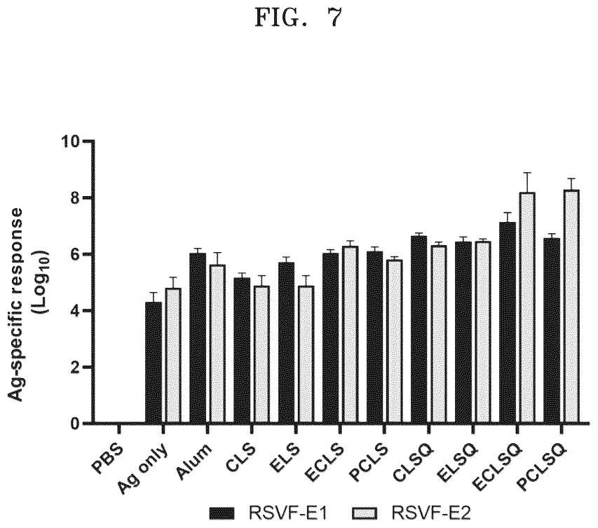

As a result, as shown in FIG. 7, compared to the test group administered with the antigen (RSVF-E1 and RSVF-E2) alone, antibody titers were found to increase in the test groups administered with the antigen in combination with liposomes or alum. The antibody titer was found about 10 times higher in the test group administered with ELS in which the antigen was mixed with the liposome formulation, compared to the test group administered the antigen alone. Compared to the ELS-administered group, an increased antibody titer was found in the ECLS-administered group, thereby indicating that vaccine efficacy may be increased by adding CoPoP that enhances the ability to present antigen. Antibody titer in the ECLSQ-administered group was found about 100 times to 1,000 times higher than that in the ELS- and ECLS-administered groups, thereby showing that QS-21 may induce enhancement of humoral immunity as an immune adjuvant. Upon comparison between the CLS and ECLS test groups and between the CLSQ and ECLSQ test groups, addition of monophosphoryl lipid A as the immune adjuvant was found to have enhanced the ability to induce antibody production. In addition, although there was no significant difference in the ability to induce humoral immunity between the antigen candidates, RSVF-E1 and RSVF-E2, the RSVF-E2 repeatedly showed slight superiority.

It was confirmed that the liposome formulation including the RSVF-E2 antigen, monophosphoryl lipid A, and CoPoP was effective in inducing production of antibodies against the RSV antigen, and that the ECSLQ formulation prepared by combining the liposome formulation with QS-21 significantly enhanced production of antibodies against the antigen.

5.3. Measurement of RSV-specific Neutralizing Antibody Titer

In order to measure a titer of an antibody specifically neutralizing infection by RSV in a mouse serum after immunization using the vaccine composition, a microneutralization (MN) assay was used. The titer of the neutralizing antibody was measured by cytopathic effect (CPE) by the virus according to dilution of serum.

Mouse serum obtained after second immunization as described in Section 5.1 was reacted at 56° C. for 30 minutes to inactivate any potentially disturbing substances such as a complement. Subsequently, the serum was serially diluted (2-fold dilution) using a MEM medium supplemented with 2% fetal bovine serum (FBS) and 150 μl of the dilution was aliquoted into each well of a separate 96-well plate. A starting dilution concentration may vary according to an expected range of neutralizing antibody titer. 150 μl of the virus (RSV A2(ATCC, VR-1540) or RSV 18537(ATCC, VR-1580)) diluted to 103 TCID50/ml was aliquoted into the plate containing the serum and incubated in an incubator at 37° C. for 1.5 hours. 100μ of the collected mixture was added to Hep-2 cells aliquoted into a 96-well plate prepared on the previous day and incubated in an incubator at 37° C. for 5 days. On Day 5, the cell medium was removed from the plate, and 3-(4,5-dimethylthiazol-2-yl)-2,5-diphenyltetrazolium bromide solution was added thereto at 1 mg/mL to determine cell viability.

An inhibition concentration 50 (IC50) at which virus infection is inhibited by 50% was calculated based on cell viability obtained by the MN assay by using a negative or positive control. The inhibition concentration 50 (IC50) at which virus infection is inhibited by 50% was calculated by using “Reed-Muench method”.

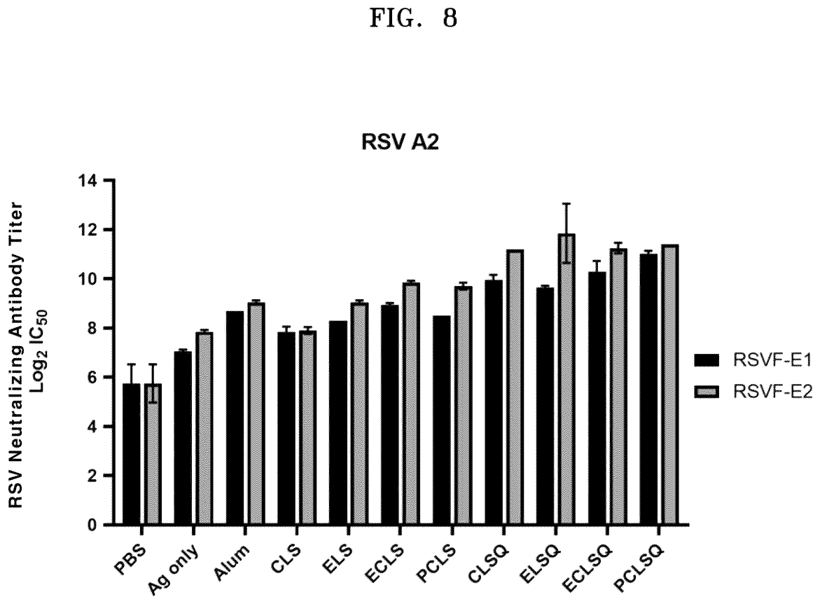

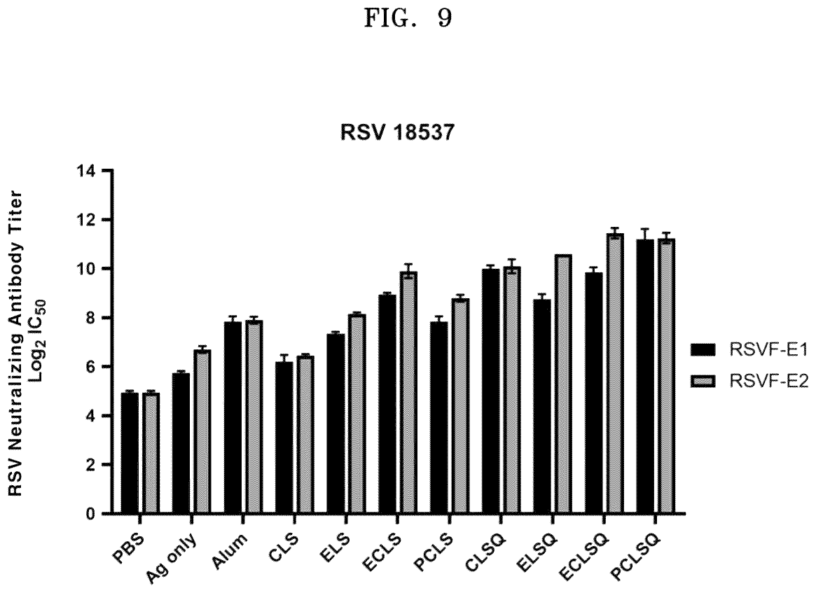

As a result, as shown in FIGS. 8 and 9, it was found that the neutralizing antibody titer for RSV A2 or RSV 18537 increased in the test groups using the immune adjuvants compared to the group administered with the antigen alone. Increase in the neutralizing antibody titer by the combination with the immune adjuvants was found identical to that of the antigen-specific serum antibody titer as described above.

It was confirmed that the ELS-administered group showed a higher neutralizing antibody titer than that of the group administered with the antigen alone, and the group administered with ECLS, which further comprises CoPoP, showed a higher neutralizing antibody titer than that of the ELS-administered group. In addition, in the test group administered with ECLSQ, which was prepared by adding QS-21 to ECLS, the highest level of neutralizing antibody titer was found. Also, the neutralizing antibody titers of the groups administered with CLS, ECLS, CLSQ, and ECLSQ showed that monophosphoryl lipid A induced enhancement of humoral immunity. Further, the RSVF-E2 antigen was found superior to the RSVF-E1 antigen in inducing neutralizing antibody against RSV.

The liposome formulation including the RSVF-E2 antigen, monophosphoryl lipid A, and CoPoP (ECLS) and the combination of the liposome formulation and QS-21 (ECLSQ) were found significantly effective in inducing neutralizing antibody of RSV.

5.4. Measurement of RSV-Specific Cell-Mediated Immunity

In order to identify T cells specifically activated by RSV, levels of interferon gamma (IFN-gamma) were measured. Interferon gamma is a type of cytokine secreted by T cells that respond specifically to antigens and is used as a representative indicator when cell-mediated immunity for antiviral activity is measured.

First, 3 weeks after administration of the vaccine composition twice as shown in Section 5.1, two mice per group were sacrificed and spleens were removed therefrom. The spleen was placed on a 40 μm-mesh, crushed with a syringe plunger, and washed with a RPMI1640 medium. The spleen from each group was centrifuged at 500×g for 5 minutes at 4° C., and a RBC lysis buffer (0.083% ammonium chloride in 0.01 M Tris buffer) was added to remove erythrocytes. After centrifugation and final washing, the resultant was resuspended in Complete RPMI1640 (10% FBS, 1% Antibiotics), and the cells were counted, and 1×10⁶ cells were added to each well of a U-shaped 96-well plate.

In order to activate T cells in an antigen-specific manner, the isolated splenocytes were restimulated with 2 μg/ml peptides from F antigen of RSV, followed by culturing for 48 hours at 37° C. under 5% CO₂ conditions. Upon completion of culture, the resultant was centrifuged at 500×g for 5 minutes at 4° C., and a supernatant was collected to measure cytokine secretion. Secretion of interferon gamma of each group was analyzed by Cytokine ELISA (R&D systems) according to manufacturer's instructions.

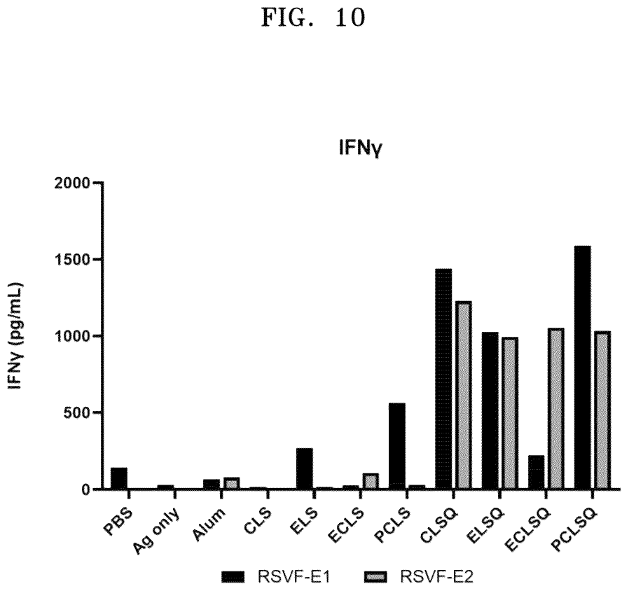

As shown in FIG. 10, it was found that the secretion of interferon gamma increased in the test groups to which the immune adjuvants were added compared to the PBS-administered group and the test group administered with the antigen alone (Ag only). Unlike the antigen-specific antibody titer and the RSV-specific neutralizing antibody titer shown in Sections 5.2 and 5.3 described above, addition of CoPoP and monophosphoryl lipid A were found not to enhance cell-mediated immunity. While the antigen-specific antibody titer was significantly increased when CoPoP was added to the antigen, the cell-mediated immunity was almost similar to that before the addition of CoPoP. These results indicate that the excellent antigen-presenting ability of CoPoP mainly promotes differentiation of B cells. On the contrary, it was found that addition of QS-21 significantly increased the secretion of interferon gamma compared to the test groups to which QS-21 was not added.

The test results of the antigen-specific antibody titer, the neutralizing antibody titer, and the cell-mediated immunity as above show that excellent humoral and cellular immunity may be efficiently induced by using the RSVF-E2 antigen, monophosphoryl lipid A, CoPoP, and/or QS-21 together.

5.5. Identification of Protective Activity Against RSV Challenge

In order to identify protective activity against RSV in the mice after immunization by the vaccine composition as described above in Section 5.1, a live-virus challenge test was performed on the mice.

The mice were divided into a PBS-administered group as a negative control and groups administered with vaccine composition including the RSVF-E2 antigen, ECLS, and QS-21 (ECLSQ), and the mice were administered therewith twice. 3 weeks after the second administration, infection was induced by injecting 10⁶ PFU of RSV through the nasal cavity. 4 days after the infection, the mice were sacrificed and lungs were removed. The lung was placed on a 40 μm-mesh, crushed with a syringe plunger, and recovered with a RPMI1640 medium. The solution of the crushed lung was centrifuged at 800×g for 10 minutes at 4° C., and a supernatant was collected therefrom. The supernatant was added to Hep-2 cells cultured in a 24-well plate, and infection was induced for 2 hours at 37° C. under 5% CO₂ conditions. Then, the supernatant was removed and an overlay MEM was added thereto, and then the cells were cultured for 7 days at 37° C. under 5% CO₂ conditions. After culturing, living cells were stained with a crystal violet solution, and the virus proliferated in the lung was quantified by counting plaques formed by the virus.

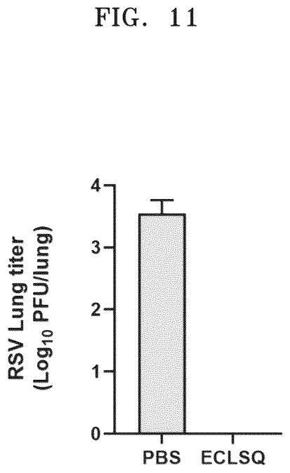

The results are shown in FIG. 11. In the group administered with the vaccine composition including the RSVF-E2 antigen, ECLS, and QS-21, proliferation of RSV was inhibited in the lung tissue. On the contrary, proliferation of RSV was found in the PBS-administered group, a negative control. This indicates that administration of the vaccine composition including the RSVF-E2 antigen, ECLS, and QS-21 induces protective activity against RSV in mice.

Overall, it was found that excellent humoral and cellular immunity can be induced by using the combination of the RSVF-E2 antigen, monophosphoryl lipid A, CoPoP, or QS-21 compared to the group administered with the RSVF-E2 antigen alone. Further, it was found that humoral and cellular immunogenicity were different depending on components of the vaccine composition, and that the vaccine composition prepared by adding ECLS and QS-21 to the RSVF-E2 antigen had excellent protective activity in the RSV challenge test in mice.

5.6. Identification of Occurrence of VAERD by Vaccine Composition

In order to identify whether immunogenicity induced by RSV vaccination causes a vaccine-associated enhanced respiratory disease (VAERD) from natural infection by RSV, a live-virus challenge test was performed after immunization by the liposome formulation (ECLS) including the RSVF-E2 antigen, monophosphoryl lipid A, and CoPoP, and by the RSV vaccine composition including the combination of the liposome formulation and QS-21 (ECLSQ or ECLS), followed by histopathological analysis in the lung. The VARED refers to worsening of respiratory symptoms when infected with RSV after vaccination due to an intended immune response by vaccination.

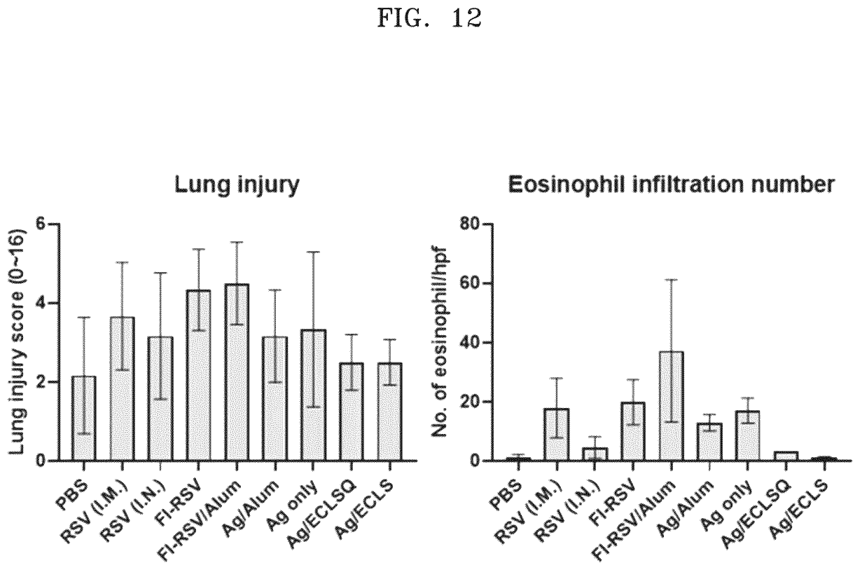

As shown in FIG. 12, the formalin-inactivated respiratory syncytial virus (FI-RSV) or a combination thereof with alum (FI-RSV/Alum) set as positive controls for VAERD led to high levels of “lung injury” and “eosinophil” in all test groups, indicating that VAERD as a side effect of the vaccination was normally induced in the test. On the contrary, in the group administered with the vaccine composition including the RSVF-E2 antigen, ECLS, and QS-21 (Ag/ECLSQ) and the group administered with the vaccine composition including the RSVF-E2 antigen and ECLS (Ag/ECLS), the levels of “lung injury” and “eosinophil”, used as direct indicators of vaccine-associated enhanced respiratory disease (VAERD) the lung, were lower than those of the other controls. These results indicate that the RSV vaccine compositions (ECLSQ and ECLS) according to embodiments of the present disclosure have low probability of causing the vaccine-associated enhanced respiratory disease (VAERD).

Because the vaccine composition according to an embodiment of the present disclosure includes a liposome formulation including monophosphoryl lipid A derived from recombinant E. coli (EcML) and a cobalt porphyrin phospholipid (CoPoP) capable of making the RSV recombinant protein antigen (RSVF-E2) binding to the surface of the liposome formulation to increase absorption of the antigen into the antigen-presenting cell (APC), excellent vaccine efficacy is provided thereby. Therefore, the vaccine composition may be used as an effective RSV vaccine in preventing or treating a disease caused by RSV infection.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A vaccine composition for preventing respiratory syncytial virus (RSV) infection, which is in the form of a liposome formulation comprising a RSV antigen, monophosphoryl lipid A (MLA), and/or a cobalt-porphyrin-phospholipid (CoPoP) conjugate.

2. The vaccine composition of claim 1, further comprising QS-21.

3. The vaccine composition of claim 1, wherein the RSV antigen is a RSVF-E2 (respiratory syncytial virus fusion glycoprotein E2) antigen.

4. The vaccine composition of claim 1, wherein the RSVF-E2 antigen has an amino acid sequence of SEQ ID NO: 1.

5. The vaccine composition of claim 1, wherein the vaccine composition is a liposome formulation comprising EcML derived from E. coli as the MLA, cholesterol, a phospholipid, and CoPoP.

6. The vaccine composition of claim 1, wherein the RSV antigen comprises a polyhistidine tag.

7. The vaccine composition of claim 6, wherein the polyhistidine tag comprises 5 to 10 histidine residues.

8. The vaccine composition of claim 6, wherein at least a portion of the polyhistidine tag is present in a hydrophobic portion of a monolayer or bilayer of the liposome, at least one histidine residue of the polyhistidine tag forms a coordinate bond with cobalt of the CoPoP, and at least a portion of the RSV antigen is exposed to the exterior of the liposome.

9. The vaccine composition of claim 1, further comprising an additional immune adjuvant.

10. The vaccine composition of claim 1, further comprising an additional RSV antigen.

11. A method of preparing a vaccine composition for preventing respiratory syncytial virus (RSV) infection, the method comprising:
culturing host cells transfected with an expression vector including a gene encoding a RSVF-E2 (RSV fusion glycoprotein E2) antigen and having a nucleotide sequence of SEQ ID NO: 2; and
obtaining the RSVF-E2 antigen comprising an amino acid sequence of SEQ ID NO: 1 from a culture of the host cells.

12. The method of claim 11, wherein the obtaining the RSVF-E2 antigen comprises purifying the culture of the host cells by affinity chromatography to obtain a first eluate, purifying the first eluate by anion exchange chromatography to obtain a second eluate, purifying the second eluate by cation exchange chromatography to obtain a third eluate, and purifying the third eluate by tangential flow filtration (TTF) to obtain a RSVF-E2 antigen.

13. The method of claim 11, wherein the host cells are CHO cells.

14. The method of claim 11, wherein the RSVF-E2 antigen comprises a polyhistidine tag at a C-terminal.

15. The method of claim 11, wherein the vaccine composition is a liposome formulation, and the method further comprises preparing a liposome formulation including MLA or CoPoP by adding MLA or CoPoP to a composition for preparing a liposome including a phospholipid and cholesterol.

16. The method of claim 15, further comprising mixing the RSVF-E2 antigen with the liposome formulation and mixing the liposome formulation with QS-21.

17. The method of claim 15, wherein the preparing the liposome formulation comprises adding CoPoP or MLA to the composition for preparing a liposome to prepare a liposome formulation including the MLA and the CoPoP.

18. The method of claim 17, wherein the method further comprises mixing the RSVF-E2 antigen with the liposome formulation to make the RSVF-E2 antigen binding to the liposome formulation, and mixing QS-21 with the liposome having the RSVF-E2 antigen bound thereto.
